(19) United States
(12) Patent Application Publication
(10) Pub. No.: US 2019/0125277 A1
(43) Pub. Date: May 2, 2019

(54) USER INTERFACE OF A MEDICAL DIAGNOSIS SYSTEM, AND COMPUTER PROGRAM THEREFOR

(71) Applicant: Oliver C. RADKE, Bremerhaven (DE)

(72) Inventor: Oliver C. RADKE, Bremerhaven (DE)

(21) Appl. No.: 16/096,336

(22) PCT Filed: Apr. 19, 2017
(86) PCT No.: PCT/EP2017/059295

§ 371 Date: Oct. 25, 2018

(30) Foreign Application Priority Data
Apr. 25, 2016 (DE) 10 2016 107 603.1

(51) Int. Cl. A61B 5/00; A61B 5/053; A61B 5/08; A61M 16/00

(52) U.S. Cl. CPC A61B 5/7425; A61B 5/0536; A61B 5/0809; A61B 5/4836; A61M 2205/505; A61M 2230/65; A61M 2205/502; A61M 2230/46; A61M 16/026

(57) ABSTRACT

The invention relates to a user interface of a medical diagnosis system that has at least one evaluation computer and data output means for outputting data to a user in the form of at least one image display device, wherein the user interface can be supplied in real time with EIT data from an electrical impedance tomography (EIT) system in operation on a patient and with ventilation data from an artificial respiration system in operation on the patient, wherein the user interface is in the form of a clinical user interface that is configured, by virtue of its evaluation computer, for real-time data processing of supplied EIT data and ventilation data, wherein the user interface is configured, by virtue of its evaluation computer, to take the supplied EIT data and ventilation data for the different lung areas of the patient that are recorded by the EIT system as a basis for respectively establishing whether there is a volume trauma, an atelaclasis or a normal functional state of the lung in the respective lung area, and to graphically represent this state, so as to be distinguishable from other states, on the image display device in real time for the different lung areas on the basis of graphical features of the representation that denote the respective state. The invention further relates to a computer program that is configured to perform the functions of the user interface.

The invention relates to a user interface of a medical diagnostic system comprising at least one evaluation computer and data output means in the form of at least one image display appliance for outputting data to a user. Real-time EIT data are suppliable to the user interface from an electrical impedance tomography (EIT) system in operation on the patient. The invention further relates to a computer program configured to carry out the functions of the user interface.

In general, the invention relates to the field of electrical impedance tomography (EIT) and the clinical preparation and representation of the measurement results obtained therewith. Electrical impedance tomography, also referred to as EIT, is a non-invasive, radiation-free method for tomographic visualization of the content of the thorax. In the process, electrical signals, e.g., high-frequency alternating currents, are guided into the body by way of an electrode belt placed against the chest and the electrical impedances between different electrodes are measured. Then, it is possible to draw conclusions about the lung tissue and the air distribution in the thorax from these data. Consequently, EIT allows a non-invasive real-time measurement of the regional ventilation distribution in the lung in the case of bedridden patients.

By way of example, the PulmoVista 500 EIT system by Dräger is known; it comprises a user interface in which the regional ventilation of the lung can be visualized in cross section on an image display appliance as measurement results of the EIT system. It is also possible to reproduce further representations, in the form of time curves, for example. The representation of the data is predominantly technical, which can also be traced back to the fact that the previous use of EIT was mainly in research.

Merging and evaluating data from different systems, for example for identifying and graphically illustrating instances of regional overdistention and atelectasis of the lung, by means of scientific, experimental software called EIDORS (www.eidors.org) has already also been proposed in the research aspect of the field of EIT. A proposition to this effect was made in the paper “A Unified Approach for EIT Imaging of Regional Overdistention and Atelectasis in Acute Lung Injury”, Gómez-Laberge, Arnold, Wolf, IEEE Transactions on Medical Imaging, volume 31, no. 3, March 2012, pages 834 to 842.

However, such approaches relate to pure research applications; therefore, EIDORS software is only designed for offline applications.

The invention is based on the object of specifying an improved user interface of a medical diagnostic system, which is better suited to clinical use and which produces representations with an improved significance for clinical applications. Further, a computer program suited to this end should be specified.

This object is achieved by a user interface of a medical diagnostic system comprising at least one evaluation computer and data output means in the form of at least one image display appliance for outputting data to a user, wherein EIT data from an electrical impedance tomography (EIT) system in operation on a patient and ventilation data of a ventilator system in operation on the patient are suppliable to the user interface in real-time, wherein the user interface is configured by its evaluation computer to process the data of supplied EIT data and ventilation data in real-time, wherein the user interface is configured by its evaluation computer to determine, from the supplied EIT data and ventilation data, whether there respectively is overdistention, atelectasis or a normal functioning state of the lung in the different lung areas of the patient detected by the EIT system and to represent this state graphically, in a manner distinguishable from other states, in real time on the image display appliance for the different lung areas on the basis of graphic features of the representation characterizing the respective state.

The invention renders it possible to merge and evaluate, in real-time, the data of an EIT system and a ventilation system while these systems are in operation on the patient within the scope of clinical operation. Therefore, the user interface according to the invention can also be referred to as a clinical user interface. By way of the evaluation, there is a representation, in real-time, for the respective lung areas of the patient where overdistention, atelectasis or a normal functioning state of the lung is possibly present. In this way, the user can quickly identify problems in the ventilation and counteract these problems by modifying the adjustment parameters of the ventilation system. Ventilation protection of the lungs can be realized by the user interface according to the invention in a simplified and quicker fashion, said ventilation avoiding possible additional ventilation-associated damage to the diseased lung from the outset. By combining the EIT data with the ventilation data, it is possible to immediately identify overdistended and atelectatic regions. This is now made immediately visible to the user by the clinical user interface according to the invention.

The user interface can be embodied as a dedicated system or appliance (standalone appliance). It may also be integrated in another medical system, e.g., in an EIT system or a ventilation system. The user interface may have an EIT data interface to an EIT system and/or a ventilation data interface to a ventilation system, said interfaces respectively being embodied as real-time interfaces. The EIT data can be supplied to the user interface by the EIT data interface; the ventilation data can be supplied by the ventilation data interface. Depending on whether the user interface is integrated in an EIT system or ventilation system, for example, either the EIT data interface or the ventilation data interface can be dispensed with or can be embodied as system-internal interfaces.

The user interface may also be integrated in any other medical system, a patient monitor, for example. The other medical system can also be a combined system with an EIT functionality and a ventilation system functionality. In this case, both aforementioned data interfaces can be dispensed with or can be embodied as internal interfaces.

Further, the user interface may comprise data input means for entering data into the user interface by a user, e.g., data input means such as keyboard, mouse or touchscreen, which are known in relation to computers. In this way, the user can carry out settings on the user interface or other functions of the medical system.

According to an advantageous development of the invention, provision is made for the user interface to be configured by its evaluation computer to represent the states of the lung areas on the image display appliance in a two-dimensional representation corresponding to a sectional plane through the lungs of the patient. In this way, it is possible to reproduce the states of the lung areas in a computed-tomography-like representation, with the difference that, in contrast to the latter, the data can be detected directly on the bedridden patient and the patient is not exposed to a radiation load. In the two-dimensional representation, the lung areas identified as overdistended, atelectatic or normal may be characterized differently, by different colors, for example.

According to an advantageous development of the invention, provision is made for the user interface to be configured by its evaluation computer to additionally represent the states of the lung areas in a lung state/time diagram on the image display appliance. In this way, the user is supplied with additional helpful clinical information with a great significance.

According to an advantageous development of the invention, provision is made for the user interface to be configured by its evaluation computer to represent a time diagram, synchronous in time with the lung state/time diagram, of the ventilation pressures of the ventilation system on the image display appliance. This allows, for example, the maximum pressure occurring in a respiratory cycle and the PEEP (positive end-expiratory pressure) to be represented in such time diagrams.

According to an advantageous development of the invention, provision is made for the user interface to be configured by its evaluation computer to represent further detected parameters of the patient in a numerical representation on the image display appliance in time-synchronous fashion to the lung state/time diagram. By way of example, this allows further parameters, such as the ventilation mode employed by the ventilation system, e.g., BiPAP, the Horowitz index, CO₂ values, the tidal volume and further parameters, to be displayed.

According to an advantageous development of the invention, provision is made for the image display appliance to be embodied as a touchscreen, wherein the user interface is configured to be operated by gesture control on the touchscreen. This allows particularly simple and intuitive operation of the user interface. By way of example, scaling of the represented diagrams or displacement by drag-and-drop inputs may be provided. Scaling can be carried out by pinch gestures.

According to an advantageous development of the invention, provision is made for the user interface to have one or more expert systems, which are activatable by a user by way of a data input means of the user interface, wherein a respective expert system is configured for a certain course of treatment for the patient and said expert system has a user guidance according to a question-answer system, by means of which the user is systematically guided through individual steps of the respective course of treatment. In this way, the user can be provided with additional assistance that is directly integrated into the user interface or the medical diagnostic system. Consequently, the user need not commit to memory every detail in relation to the respective newest prior art within a multiplicity of possible courses of treatment. The user can be systematically guided through the individual steps of the respective course of treatment by way of an appropriate user guidance according to the question-answer system. By way of example, an expert system for PEEP trial, for recruitment therapy and/or for therapeutic bedding may be implemented.

In the PEEP trial, the most advantageous PEEP for the patient is searched for. To this end, for example, the expert system can instruct the user to set and trial various pressures on the ventilation system. The corresponding evaluation of the reactions of the patient can be implemented by the user interface, and so the expert system can immediately propose to the user appropriate further steps of the treatment.

Accordingly, the instructions output to the user are determined automatically depending on the clinical evaluation of the EIT data fed to the user interface and the ventilation data, and optionally on data of further connected systems.

Within the scope of recruitment, the user can be provided with instructions for setting the ventilation appliance, which are helpful for reopening collapsed lung regions. In the case of therapeutic bedding, the user can be provided with instructions relating to how the patient should be re-bedded at what time, for example in order to avoid the formation of edemas.

According to an advantageous development of the invention provision is made for the user interface to be configured by its evaluation computer to classify the states of the lung areas, in each case according to the three discrete states of overdistention, atelectasis and normal. This frees the user from deciding what numerical parameters constitute an abnormal state of a lung area. A clear representation is facilitated by the subdivision into three discrete states. The representation of the different states of the lung areas can be implemented by different image patterns or textures and/or by different colors, for example, on the image display appliance.

According to an advantageous development of the invention, provision is made for the user interface to be configured by its evaluation computer to represent a real-time representation of the ventilation of the lung (tidal image) on the image display appliance. In this way, as it were, the raw data of the electrical impedance tomography are represented without diagnostic assessment on the basis of the ventilation data. In this way, the user is provided with additional helpful clinical information with great significance.

According to an advantageous development of the invention, provision is made for the user interface to be configured by its evaluation computer to represent the overdistention of the lung on the image display appliance, depending on the maximum value of the air pressure in the lung, for example in a separate diagram. By way of example, this display representation can be updated when the representation is touched by the user on a touchscreen. Thereupon, an evaluation program (wizard) is started in order to systematically determine the curve (overdistention of the lung depending on the maximum value of the air pressure in the lung).

According to an advantageous development of the invention, provision is made for the user interface to be configured by its evaluation computer to represent the atelectasis formation of the lung on the image display appliance, depending on the PEEP, for example in a separate diagram. By way of example, this display representation can be updated when the representation is touched by the user on a touchscreen. Thereupon, an evaluation program (wizard) is started in order to systematically determine the curve (atelectasis formation of the lung depending on the PEEP).

According to an advantageous development of the invention, provision is made for the user interface to be configured by its evaluation computer to represent a plurality of collected two-dimensional representations of the states of the lung areas from the past on the image display appliance. By way of example, these representations can be reproduced as miniatures of the real-time representation of the states of the lung areas. In this way, a relatively large number of past images of the states of the lung areas can be reproduced in the case of limited spatial requirements on the image display appliance. The representation can be updated synchronously in time with the remaining representations. By touching the touchscreen in the region of this representation and by swiping thereover, it is possible to show individual images from the flow of the plurality of collected two-dimensional representations.

According to an advantageous development of the invention, provision is made for the user interface to be configured by its evaluation computer to represent diagrams with SpO₂, CO₂ and/or Horowitz index data on the image display appliance. This additionally improves the meaningfulness of the data reproduced on the image display appliance for the clinical user.

To this end, data of further systems can be supplied to the user interface as well, e.g., data of an SpO₂ monitor, data of a CO₂ monitor, with these data also being able to come from the ventilation appliance, and data from a blood gas analysis laboratory.

Accordingly, the user interface may have further data interfaces, e.g., to an SpO₂ monitor, a blood gas analysis laboratory or any other equipment for patient monitoring. In this way, the user interface can be linked, in particular, to a laboratory system, e.g., a blood gas analysis appliance, in order, for example, to display the arterial oxygen partial pressures in conjunction with the remaining represented data. The Horowitz index, in particular, can be determined from the data provided by the blood gas analysis appliance.

According to an advantageous development of the invention, the user interface can be configured by its evaluation computer to represent on the image display appliance an indication of the signal quality of the measurement signals recorded by the EIT system. This allows a fast assessment of the reliability of the remaining represented data.

The explained types of representation of the data on the image display appliance, e.g., the diagrams, can be represented individually or, in combination together, at the same time on the image display appliance. In an advantageous configuration of the invention, the user interface is configured to represent different combinations of representations, for which dedicated image combination patterns (screens) are programmed in each case. In an advantageous configuration of the invention, the user interface has a dashboard screen, which is a type of basic display mode of the data output by the user interface on the image display appliance. It is possible to switch over to other representations from the dashboard screen, for example by virtue of one of the diagrams being selected by touch, the diagram then being represented as a full-screen image, for example. Renewed touch of the image representation leads back to the dashboard screen. By way of example, all of the aforementioned image representations and diagrams of the data may be contained on the dashboard screen, or else only a subset thereof. In particular, the dashboard screen can be embodied to be configurable such that the user can set the representations and diagrams displayed on the dashboard screen.

By way of example, the time diagrams can be embodied as time diagrams migrating continuously or at discrete time intervals over the image display appliance and/or as time diagrams with the migrating cursor.

According to an advantageous development of the invention, the diagnostic system comprises an EIT system with a plurality of electrodes to be attached around the thorax of a patient and at least one control and evaluation unit that is connected or connectable to the electrodes, said control and evaluation unit being configured to carry out an electrical impedance tomography method by way of electrical signals transmitted by the electrodes, wherein the EIT system has at least one position sensor device, which is configured to be attached to the patient in order to transmit position signals, detected at the patient, to the control and evaluation unit, wherein the control and evaluation unit is configured to determine the spatial position of the patient from the position signals, at least in one spatial dimension. This is advantageous in that the position sensor device and the position signals determined therewith can be used to improve the evaluation of the EIT signals and the diagnoses established therefrom. In the case of the output EIT images, a relationship to the actual position of the patient can be established by technical means, and so it is possible to determine at all times, both during the real-time assessment of the EIT images and when assessing recorded data at a later time, whether the patient was lying on the back or on the stomach, for example. This renders it possible to detect and represent the EIT data, in particular the changes in the ventilation distribution of the lung emerging therefrom and regional compliance changes, and to correctly interpret these changes, specifically in relation to the actual position of the patient.

As a result of the action of gravity, atelectasis, for example, may arise in the lung, especially in the depending parts of the lung. Patients only lying in supine position in the bed therefore have a high risk of a formation of atelectasis in the dorsal region, especially if damage to the lung is already present. Therefore, it is recommended that patients in intensive care be bedded according to rules, e.g., in alternating lateral positions or even in a prone position, in order to reduce the formation of atelectasis. As a result of this bedding therapy, there is a change in the gravity vector acting on the patient, and so the formation of atelectasis can be reduced. The invention renders it possible to correctly interpret the recorded EIT data at all times, even in the case of such a bedding therapy, specifically by taking account of the actual bedding or position of the patient.

As a result of the additional information available from the position sensor device, the intensive care physician can recognize what effects the different bedding types have had on the lung and may draw conclusions therefrom for further therapy planning.

The at least one spatial dimension of the spatial position of the patient, which is intended to be determined, may be the rotational angle about the longitudinal axis of the patient, for example. If use is made of a multi-axis position sensor device, the spatial position of the patient, e.g., the inclination of the patient in the longitudinal direction, can even be determined in a plurality of spatial dimensions from the position signals of said position sensor device, at least in the region of the thorax.

According to an advantageous development of the invention, provision is made for the control and evaluation unit to be configured to represent the position of the patient, determined from the position signals, on the basis of a position indicator that is output on an image display appliance. Thus, the position of the patient can be output on the image display appliance, for example, in the form of numerically output angle values of the angle position or by way of a graphical symbol, e.g., an arrow with an arrow direction that is set depending on the angle position. This facilitates a simple and quick assessment of the EIT data represented on the image display appliance in relation to the actual position of the patient.

According to an advantageous development of the invention, provision is made for the control and evaluation unit to be configured to apply an electrical feed signal to at least one electrode pair as a feeding electrode pair and to record an electrical measurement signal with a plurality of the remaining electrode pairs in each case, and to successively let other electrode pairs act as feeding electrode pairs in order to reconstruct a matrix of image elements from the plurality of electrical measurement signals using a reconstruction algorithm, said matrix representing the distribution of the impedance changes in the electrode plane, and to repeatedly carry out such processes over time and to reconstruct matrices herefrom and to represent the the matrices on an image display appliance. By means of such matrices, it is possible to represent, e.g., a so-called tidal image of the lung function. This helps the user to make a diagnosis.

According to an advantageous development of the invention, provision is made for the control and evaluation unit to be configured to represent the matrices on an image display appliance taking account of the position of the patient as determined from the position signals. Thus, the matrix represented on the image display appliance may be represented, for example, in a rotated fashion corresponding to the angle position of the patient; i.e., in the case of a patient lying on their stomach, the matrix is represented rotated through 180° in comparison with a patient lying on their back.

The object specified at the outset is further achieved by a computer program, configured to carry out the functions of the user interface of the type set forth above when the computer program is executed on the evaluation computer of the user interface. The advantages explained above can also be obtained hereby.

Below, the invention will be explained in more detail on the basis of exemplary embodiments, with reference being made to drawings. In the drawings:

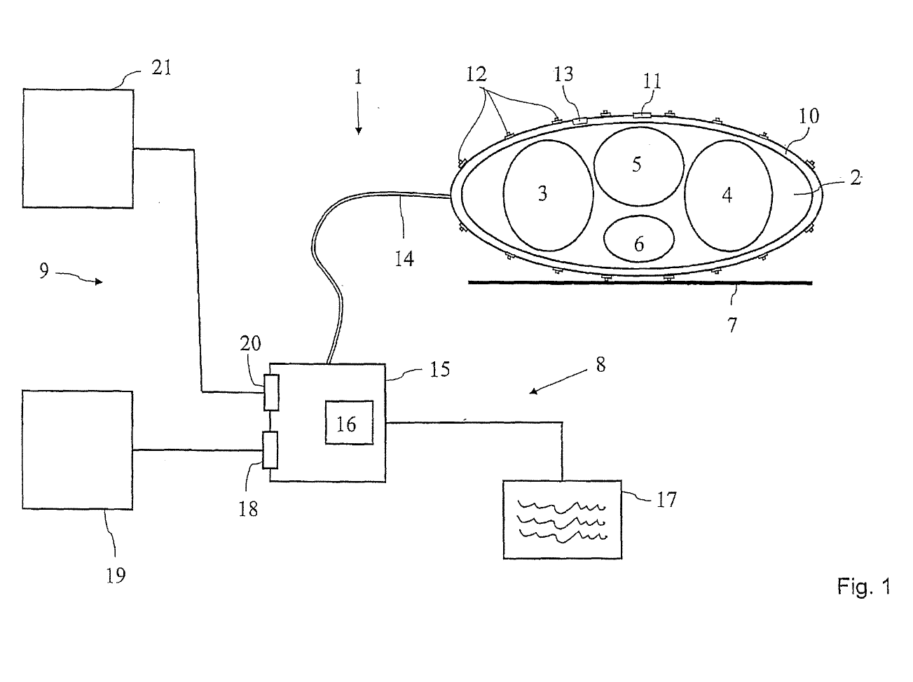

FIG. 1 shows a medical diagnostic system and

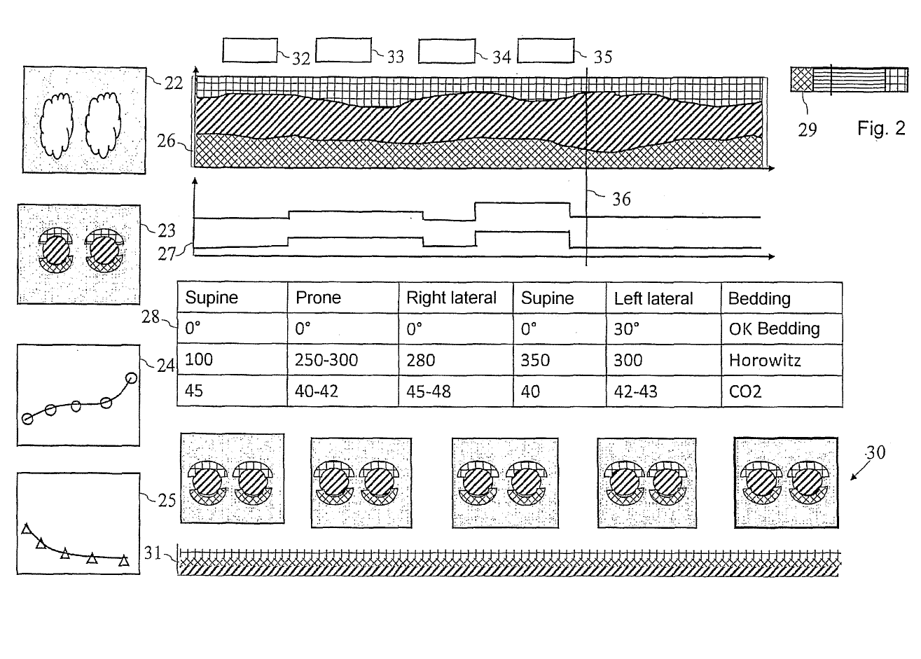

FIG. 2 shows a dashboard screen on the image display appliance of the medical diagnostic system

FIG. 1 shows a cross-section of a patient 2, or their thorax, lying on a horizontally aligned support 7. The lungs 3, 4, the heart 5 and the spinal column 6 are identifiable.

Further, a diagnostic system 9 with an EIT device 1 is represented, said EIT device having a control and evaluation unit 15 and an electrode belt 10. The electrodes 12 of the EIT device, which serve to detect the impedance values during the electrical impedance tomography, are arranged or worked into the electrode belt 10. The electrode belt 10 can be opened at a closure point 11 and be placed around the patient 2 in the opened state. The electrode belt 10 is then closed at the closure point 11 and rests tightly around the thorax on the patient, for example level with the fifth intercostal space, as is identifiable in FIG. 1. The electrodes 12 are connected to a connection cable 14 via electric lines that have been routed through the electrode belt 10, the electrode belt 10 or the electrodes 12 thereof, being electrically connected to the control and evaluation unit 15 by way of said connection cable.

The electrode belt 10 additionally has a position sensor device 13, for example in the form of an acceleration sensor, which is arranged at the electrode belt or integrated therein. The position sensor device 13 is likewise connected to the control and evaluation unit 15 via an electric line that is guided through the electrode belt 10 and the cable 14. In this way, the position signals of the position sensor device 13 are electrically transmitted to the control and evaluation unit 15.

The control and evaluation unit 15 has a computer 16, for example a micro processor, which controls the signal feed of the electrical signals of the electrical impedance tomography into the patient 2 and which moreover evaluates and processes the received signals of the electrodes 12 and of the position sensor device 13, for example by way of appropriate software programming of the computer 16.

As may be identified, the electrode belt 10 has a plurality of electrodes, wherein 16 or 32 electrodes, for example, may be present. In each measurement cycle, respectively two of the electrodes 12 are used to feed a high-frequency alternating current into the patient, for example at a frequency in the range from 5 to 500 kHz and a current of at most 5 mA_{eff}. The arising potential differences are measured successively at the, e.g., 13 other adjacent electrode pairs. Then, the electrical signal feed is displaced by one electrode 12 and the potential difference is measured anew at all other electrode pairs. This is repeated in rotation until all electrode pairs have been used once for the feed. A two-dimensional image representation, e.g., a matrix of 32×32 impedance values, which represent the cross section of the thorax under the electrode belt 10, is produced from the multiplicity of measurement values obtained in the process by way of evaluation programs, as are implemented, for example, in the PulmoVista 500 system by Drager. This process is repeated. The sequence of the matrices determined in the process is stored in the EIT device and is available for further analyses and data export.

The matrices obtained in this manner, which are representable as an image, can be represented by the control and evaluation unit 15, for example on an image display appliance 17 connected thereto. Additionally, other curves, e.g., of data of the ventilation, can also be represented.

For the recorded matrices, for example for each recorded matrix or at greater time intervals, the control and evaluation unit 15 detects the respective spatial position of the patient 2 in respect of the angle position about the longitudinal axis of the patient on the basis of the position signals output by the position sensor device 13. The spatial position detected in this way can be stored in units of degrees, for example, and can be displayed in the user interface of the EIT device in conjunction with the electrical impedance tomography data or the matrices on the image display appliance 17.

Moreover, the diagnostic system 1 has a user interface 8, by means of which a user of the diagnostic system 1 can operate the latter and by means of which data detected by the diagnostic system are displayed to said user. The user interface 8 is formed at least by an evaluation computer and an image display appliance, in combination with the user interface software implemented by the evaluation computer. The image display appliance 17 may serve as image display appliance of the user interface 8; the computer 16 of the control and evaluation unit 15 may serve as evaluation computer. However, the user interface can also be embodied separately from the EIT device 1 and then it is connected via appropriate interfaces to the EIT device 1 and further systems. Data input means may also belong to the user interface 8. In the exemplary embodiment, the assumption is made that the image display appliance 17 has a touchscreen, and so inputs of the user can be implemented by way of the touchscreen.

In order to realize the invention, the EIT data must be combined with ventilation data of the patient 2. In the exemplary embodiment represented in FIG. 1, the EIT device 1, or the control and evaluation unit 15 thereof, is configured to this end by virtue of having a ventilation data interface 18, by means of which a data connection to a ventilation system 19 is established. The ventilation system 19 transmits the respective current ventilation data of the patient 2 to the EIT device 1 or to the user interface 8 via the ventilation data interface 18. Additionally, there may be a further data interface 20 to an SpO₂ monitor 21, via which data of the SpO₂ monitor 21 are supplied to the user interface 8.

The aforementioned functions of the control and evaluation unit 15 can also be implemented by the evaluation computer of the operator interface 8. In particular, the operator interface 8 can then represent the matrix represented on the image display appliance 17 in accordance with the angle position of the patient 2 in a rotated fashion and/or represent the position indicator.

A dashboard screen according to FIG. 2 is represented in a basic display mode of the data output by the user interface 8 on the image display appliance 17. The dashboard screen has the individual elements of the display set forth below.

A tidal image 22 is represented top left, in which a live representation of the ventilation of the lung 34 is reproduced. Below, in a comparable sectional illustration as in the tidal image 22, a ventilation distribution image 23 for the different lung areas of the patient 2 in each case represents whether there is overdistention, atelectasis or a normal functioning state of the lung in the respective lung area. By way of example, the mark in the upper region can denote an overdistended lung region and the mark in the lower region can denote an atelectatic lung region. The lung region therebetween is in the normal functional state. When the image 22 or the image 23 is touched, it is respectively enlarged to form a full-screen image. Touching the touchscreen again switches back to the dashboard.

The diagram 24 reproduces a representation of the overdistention of the lung depending on the peak pressure of the ventilation (Ppeak). When the diagram 24 is touched, a wizard is started to systematically create and update the represented curve.

The diagram 25 shows a representation of the formation of atelectasis depending on PEEP. When the diagram 25 is touched, a wizard for a PEEP trial method is started.

Functional elements 32, 33, 34, 35, which start a respective expert system when touched, said expert system being able to assist a certain course of treatment, are situated in the upper region of the dashboard.

Below, there is a time diagram 26, which, plotted over time, shows the percentage of lung components in the atelectatic, overdistended or normal functional state at the given point in time. By way of example, atelectatic conditions are reproduced in the lower region of the diagram, overdistended regions are reproduced in the upper region and the normal functional state is reproduced therebetween. By way of a cursor 36, individual times can be selected by the user and data corresponding thereto can be displayed.

Below the time diagram 26, there is a further time diagram 27, which is represented in time-synchronous manner with the time diagram 26. By way of example, the Ppeak and PEEP ventilation pressures are displayed in the time diagram 27.

There is an indicator 29, which represents the signal quality of the EIT measurement signals, top right on the dashboard screen.

Below the time diagram 27, further parameters are reproduced directly in numerical fashion over time, for example the ventilation mode of the ventilation system, the current position or bedding of the patient according to the values detected by the position sensor device, the Horowitz index, CO₂ values, tidal volume and further values.

A collection of images of the ventilation distributions 23, i.e. a plurality of values from the past, is represented therebelow as representation 30. When touching the representation 30 and swiping thereover, individual images from the flow of the reproduced images are represented in enlarged fashion. The cursor 36 runs synchronously therewith. Unlike what is reproduced in FIG. 2, the individual images can also be represented overlapping one another. In this way, even more individual images can be represented in the case of restricted spatial requirements on the dashboard.

A diagram 31 is represented therebelow, it being possible to display, e.g., SpO₂, CO₂ and Horowitz index values in said diagram. The representation is implemented in time-synchronous fashion with the upper diagrams 26, 27.

1. A user interface of a medical diagnostic system comprising:
at least one evaluation computer and data output means including at least one image display appliance for outputting data to a user, wherein electrical impedance tomography (EIT) data from an EIT system in operation on a patient and ventilation data of a ventilator system in operation on the patient are suppliable to the user interface in real-time,
wherein the user interface is configured by its at least one evaluation computer to process of supplied EIT data and ventilation data in real-time,
wherein the user interface is configured by its at least one evaluation computer to determine, from the supplied EIT data and ventilation data, whether there respectively is overdistention, atelectasis or a normal functioning state of the lung in different lung areas of the patient detected by the EIT system and to represent this state graphically, in a manner distinguishable from other states, in real time on the at least one image display appliance for the different lung areas on the basis of graphic features of the representation characterizing the respective state.

2. The user interface as claimed in claim 1 wherein the user interface is configured by its at least one evaluation computer to represent the states of the lung areas on the at least one image display appliance in a two-dimensional representation corresponding to a sectional plane through the lungs of the patient.

3. The user interface as claimed in claim 1 wherein the user interface is configured by its at least one evaluation computer to represent the states of the lung areas in a lung state/time diagram on the at least one image display appliance.

4. The user interface as claimed in claim 3 wherein the user interface is configured by its at least one evaluation computer to represent a time diagram, synchronous in time with the lung state/time diagram, of the ventilation pressures of the ventilation system on the at least one image display appliance.

5. The user interface as claimed in claim 3 the user interface is configured by its at least one evaluation computer to represent additional detected parameters of the patient in a numerical representation on the image display appliance in time-synchronous fashion to the lung state/time diagram.

6. The user interface as claimed in claim 1 wherein the at least one image display appliance is embodied as a touchscreen, wherein the user interface is configured to be operated by gesture control on the touchscreen.

7. The user interface as claimed in claim 1 wherein the user interface has one or more expert systems which are activatable by a user by way of a data input means of the user interface, wherein a respective expert system of the one or more expert systems is configured for a certain course of treatment for the patient and said expert system has a user guidance according to a question-answer system, by means of which the user is systematically guided through individual steps of the respective course of treatment.

8. The user interface as claimed in claim 1 wherein the user interface is configured by its at least one evaluation computer to classify the states of the lung areas, in each case according to the three discrete states of overdistention, atelectasis and normal.

9. The user interface as claimed in claim 1 wherein the user interface is configured by its at least one evaluation computer to represent a real-time representation of the ventilation of the lung on the image display appliance.

10. The user interface as claimed in claim 1 wherein the user interface is configured by its at least one evaluation computer to represent the overdistention of the lung on the at least one image display appliance, depending on a maximum value of air pressure in the lung.

11. The user interface as claimed in claim 1 wherein the user interface is configured by its at least one evaluation computer to represent the atelectasis formation of the lung on the image display appliance, depending on the positive end-exiratory pressure (PEEP).

12. The user interface as claimed in claim 1 wherein the user interface is configured by its at least one evaluation computer to represent a plurality of collected two-dimensional representations of the states of the lung areas from the past on the at least one image display appliance.

13. The user interface as claimed in claim 1 wherein the user interface is configured by its at least one evaluation computer to represent diagrams with SpO₂, CO₂ and/or Horowitz index data on the at least one image display appliance.

14. The user interface as claimed in claim 1 wherein the user interface is integrated in an EIT system, a ventilation system or any other medical system.

15. A computer program encoded on a non-transitory medium, configured to carry out the functions of the user interface as claimed in claim 1 when the computer program is executable on the at least one evaluation computer of the user interface.
